(19) United States
(12) Patent Application Publication
(10) Pub. No.: US 2007/0275022 A1
(43) Pub. Date: Nov. 29, 2007

(54) Cosmetic Hair Preparation

(76) Inventors: Nobuyasu Satou, Sumida-ku (JP); Kenji Matsuda, Sumida-ku (JP)

Correspondence Address: BIRCH STEWART KOLASCH & BIRCH, PO BOX 747, FALLS CHURCH, VA, 22040-0747, US

(21) Appl. No.: 11/793,350

(22) PCT Filed: Dec. 14, 2005
(86) PCT No.: PCT/JP05/22927

§ 371 Date: Jun. 14, 2007

(30) Foreign Application Priority Data
Dec. 16, 2004 (JP) 2004-364230

(51) Int. Cl. A61K 8/37; A61K 8/34

(52) U.S. Cl. 424/401; 424/70.1

(57) ABSTRACT

One or more esters selected among an ester of a C₂₋₁₀ fatty acid with a polyhydric alcohol, an ester of a monobasic hydroxy acid with a mono- or polyhydric alcohol, and an ester of a polybasic acid with a mono- or polyhydric alcohol are incorporated into a cosmetic hair preparation comprising a crystalline compound and a solvent in which the compound dissolves. Due to this, the crystalline compound is inhibited from crystallizing/precipitating at low temperatures and the preparation can give an improved use feeling during use.

### TECHNICAL FIELD

This invention relates to cosmetic hair preparation comprising a crystalline compound, and more particularly, to a cosmetic hair preparation which is excellent in low temperature stability, is able to prevent the crystalline compound from precipitation, particularly, at low temperatures to such an extent as possible and is excellent in feeling upon using the preparation.

### BACKGROUND ART

It is known that fatty acids having odd carbon chain length and fatty alcohols having odd carbon chain length, and salts and derivatives thereof exhibit a pilatory and hair growing effect. Hair growth agents containing them as effective ingredients have been proposed (see Patent Document 1: Japanese Patent Laid-Open No, Sho 59-27809, Patent Document 2: Japanese Patent Laid-Open No. Sho 60-4113, and Patent Document 3: Japanese Patent Laid-Open No. Hei 4-5219). With the effective ingredients such as the above-described, it is general that a greater number of carbon atoms result in a lower solubility. This may lead to a disadvantage in that the pilatory and hair growth agents become clouded or permit crystals to precipitate at low temperatures. This is concerned about a lowering of the pilatory and hair growing effect. Accordingly, there is a demand for development of cosmetic hair preparation wherein crystalline compounds are kept stable under low temperature conditions.

For the techniques of stabilizing the above-mentioned crystalline compounds, there have been proposed a technique of using, along with the crystalline compound, a cation activator as a low temperature stabilizer (see Patent Document 4: Japanese Patent Laid-Open No. Hei 1-132511), a technique of using an amphoteric copolymer and a nonionic activator whose HLB value is not higher than 10 (Patent Document 5: Japanese Patent Laid-Open No. Hei 2-76805), a technique using a cyclodextrin (Patent Document 6: Japanese Patent Laid-Open No. Hei 5-194153), and a technique using a saccharide-based activator and a nonionic activator whose HLB value is not higher than 10 (Patent Document 7: Japanese Patent Laid-Open No. Hei 9-17956).

On the other hand, for an improvement of a feeling upon using the preparation, there has been proposed (Patent Document 8: Japanese Patent Laid-Open No. Hei 9-14335) a technique of using a carboxyl group-bearing monomer, and a (meth)acrylic acid ester of a long-chain alcohol having 8 to 24 carbon atoms and/or a vinyl ester of a long-chain fatty acid having 8 to 24 carbon atoms. In this connection, there is a demand for development of cosmetic hair preparation, which is further improved both in low temperature stability and in feeling upon using the preparation.

Patent Document 1: Japanese Patent Laid-Open No. Sho 59-27809

Patent Document 2: Japanese Patent Laid-Open No. Sho 60-4113

Patent Document 3: Japanese Patent Laid-Open No. Hei 4-5219

Patent Document 4: Japanese Patent Laid-Open No. Hei 1-132511

Patent Document 5: Japanese Patent Laid-Open No. Hei 2-76805

Patent Document 6: Japanese Patent Laid-Open No. Hei 5-194153

Patent Document 7: Japanese Patent Laid-Open No. Hei 9-175956

Patent Document 8: Japanese Patent Laid-Open No. Hei 9-143035

### DISCLOSURE OF THE INVENTION

### Problem to be Solved by the Invention

The invention has been made under these circumstances. And object of the invention is to provide a cosmetic hair preparation which is able to inhibit crystallization and precipitation of a crystalline compound under low temperature conditions and is excellent in feeling upon using the preparation.

### Means for Solving the Problem

We made intensive studies in order to achieve the above object and, as a result, found that by blending specific types of ester compounds, preferably two or more compounds with a cosmetic hair preparation containing a crystalline compound and a solvent capable of dissolving the crystalline compound, it is possible to inhibit from crystallization of the crystalline compounds at low temperatures, impart an excellent low temperature stability and excellent in feeling upon use. The invention has been thus completed.

Accordingly, the invention provides:

[1] A cosmetic hair preparation comprising a crystalline compound, a solvent capable of dissolving the crystalline compound, and at least one ester compound selected from the group consisting of ester compounds of fatty acids having 2 to 10 carbon atoms and polyhydric alcohols, ester compounds of monobasic oxyacids and monohydric or polyhydric alcohols, and ester compounds of polybasic acids and monohydric or polyhydric alcohols;

[2] The cosmetic hair preparation of [1], wherein the crystalline compound is selected from (i) fatty acids having 11 to 30 carbon atoms, and salts and derivatives thereof, (ii) fatty alcohols, and (iii) ether compounds of fatty alcohols and compounds having hydroxyl group; and

[3] A method for inhibiting crystallization of a crystalline compound under low temperature conditions in a cosmetic hair preparation, said method comprising blending at least one ester compound selected from the group consisting of ester compounds of fatty acids having 2 to 10 carbon atoms and polyhydric alcohols, ester compounds of monobasic oxyacids and monohydric or polyhydric alcohols, and ester compounds of polybasic acids and monohydric or polyhydric alcohols with the cosmetic hair preparation containing the crystalline compound and a solvent capable of dissolving the crystalline compound.

### BENEFITS OF THE INVENTION

According to the invention, there can be provided a cosmetic hair preparation containing a crystalline compound and a solvent capable of dissolving the crystalline compound. The cosmetic hair preparation inhibits crystallization and precipitation of the crystalline compound at low temperatures and has an excellent feeling upon using the preparation.

### BEST MODE FOR CARRYING OUT THE INVENTION

The invention is directed to a cosmetic hair preparation comprising a crystalline compound, a solvent capable of dissolving the crystalline compound, and at least one ester compound selected from the group consisting of ester compounds of fatty acids having 2 to 10 carbon atoms and polyhydric alcohols, ester compounds of monobasic oxyacids and monohydric or polyhydric alcohols, and ester compounds of polybasic acids and monohydric or polyhydric alcohols.

Examples of the crystalline compounds include (i) fatty acids having 11 to 30 carbon atoms, and salts and derivatives thereof, (ii) fatty alcohols, and (iii) ether compounds of fatty alcohols and hydroxyl group-bearing compounds.

Examples of fatty acids having 11 to 30 carbon atoms include undecanoic acid, dodecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid and the like. Examples of salts include alkali metal salts, ammonium salts, organic amine salts of the above acids.

For the crystalline compound of the invention, salts or derivatives of fatty acids having a carbon chain length of 11 to 30 carbon atoms are preferred, and salts and derivatives of fatty acids having an odd carbon chain length serving as a hair growing ingredient are more preferred. Examples of salts or derivatives include compounds represented by the following formulae (a) to (j).

(a) Monoglycerides represented by the following general formula (1) or (2). 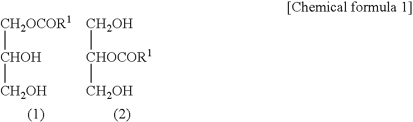
(wherein R¹ is a linear or branched monovalent hydrocarbon group having 10 to 29 even carbon atoms).

(b) Diglycerides represented by the following general formula (3) or (4). 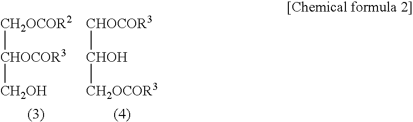
(wherein R² and R³, respectively, are a linear or branched monovalent hydrocarbon group provided that at least one thereof is a monovalent hydrocarbon group having 10 to 29 even carbon atoms, and preferably, R² and R³ are both a monovalent hydrocarbon group having 10 to 29 even carbon atoms).

(c) Triglycerides represented by the following general formula (5). 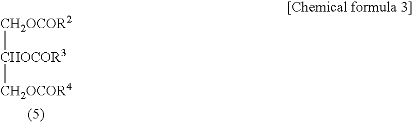
(wherein R², R³ and R⁴, respectively, are a linear or branched monovalent hydrocarbon group wherein at least one thereof is a monovalent hydrocarbon group having 10 to 29 even carbon atoms, and preferably, two or more of R², R³ and R⁴ are a monovalent hydrocarbon group having 10 to 29 even carbon atoms and more preferably, all of R², R³ and R⁴ are a monovalent hydrocarbon group having 10 to 29 even carbon atoms).

(d) Fatty acid salts represented by the following general formula (6).
(R¹COO)ₙM (6)
(wherein R¹ is a linear or branched monovalent hydrocarbon group having 10 to 29 even carbon atoms, M represents a metal atom or an ammonium ion, and n is an integer corresponding to the valence of M).

(e) Esters represented by the following general formula (7).
R¹COOR⁵ (7)
(wherein R¹ is a linear or ranched monovalent hydrocarbon group having 10 to 29 even carbon atoms, and R⁵ is a monovalent or divalent fatty acid alcohol residue, polyoxyethylene residue, sorbitan residue or sucrose residue).

(f) Amides represented by the general formulae (8 to 10). 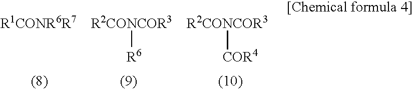
(wherein R¹ represents a linear or ranched monovalent hydrocarbon group having 10 to 29 even carbon atoms, R², R³ and R⁴, respectively, are a linear or branched monovalent hydrocarbon group provided that in the formula (9), at least one of R² and R³ is a monovalent hydrocarbon group having 10 to 29 even carbon atoms and in the formula (10), at least one of R², R³ and R⁴ is a 10 to 29 even monovalent hydrocarbon group, and R⁶ and R⁷ are a hydrogen atom, an alkyl group or a hydroxyalkyl group).

(g) Sterol esters represented by the following general formula (11). 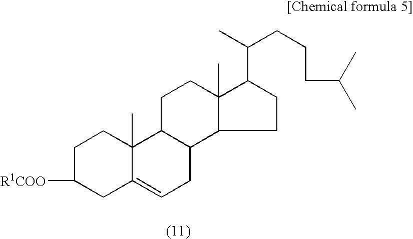
(wherein R¹ is a linear or ranched monovalent hydrocarbon group having 10 to 29 even carbon atoms).

(h) Phospholipids represented by the following general formula (12). 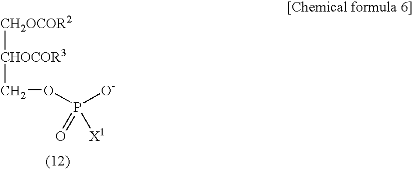
(wherein R² and R³, respectively, are a linear or branched monovalent hydrocarbon group and at least one thereof is a monovalent hydrocarbon group having 10 to 29 even carbon atoms, and X¹ is a choline residue, an ethanolamine residue, a serine residue or an inositol residue).

(i) Phosphatidic acids represented by the following general formula (13). 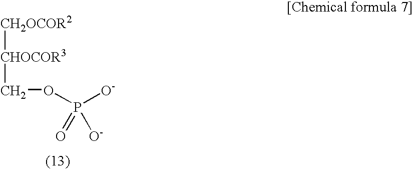
(wherein R² and R³, respectively, are a linear or branched monovalent hydrocarbon group and at least one thereof represents a monovalent hydrocarbon group having 10 to 29 even carbon atoms).

(j) Sphingolipids represented by the following general formula (14). 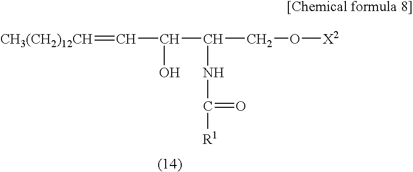
(wherein R¹ is a linear or ranched monovalent hydrocarbon group having 10 to 29 even carbon atoms, and X² is a sugar residue, a phosphoric acid residue or an amine basic residue).

The fatty alcohols used as the crystalline compound of the invention have carbon chains which may be either saturated or unsaturated, and with unsaturated chains, a plurality of double bonds may be contained. The alcohol may be either lower alcohol or higher alcohol and may be any one of primary, secondary and tertiary alcohols. For instance, the fatty alcohols include decanol, undecanol, dodecanol, tridecanol, tetradecanol, pentadecanol, hexadecanol, heptadecanol, octadecanol, nonadecanol, eicosanol, heneicosanol, docosanol, tricosanol, tetracosanol, pentacosanol and the like.

For the crystalline compound of the invention, those having a carbon chain whose number of carbon atoms is odd are preferred. Examples of fatty alcohols having odd carbon atoms include n-propyl alcohol, n-amyl alcohol, n-heptyl alcohol, n-nonyl alcohol, n-undecyl alcohol, n-tridecyl alcohol, n-pentadecyl alcohol, n-heptadecyl alcohol, n-nanodecyl alcohol, n-uneicosyl alcohol, n-tricosyl alcohol, n-pentacosyl alcohol and the like.

For derivatives of fatty alcohols, there are mentioned ether compounds of fatty alcohols and hydroxy group-bearing compounds.

Preferred ether compounds include ether compounds of such fatty acids as mentioned above, and fatty alcohols (preferably having 2 to 24 carbon atoms), polyhydric alcohols such as glycerine, polyglycerine, ethylene glycol, propylene glycol and butanediol, and sugars such as glucose, ribose, galactose, arabinose, mannose, xylose, sorbitol and mannitose. The ether compounds may contain two or more of alcohol residues such as, for example, di- or trialkoxides of glycerine. The derivatives of fatty alcohols may contain residues of such odd chain-bearing alcohols as mentioned above so far as they do not give an adverse influence on human body. Accordingly, the acid residues of the ester compounds and the alcohol residues and sugar residues of the ether compounds may be replaced by a variety of substituents.

For the crystalline compound, there may be used, aside from those compounds mentioned above, vitamins such as biotin, rivoflavin, ascorbic acid, cyanocobalamin and the like, amino acids such as glutamic acid, lysine, cysteine and the like, glutathione, quinones, and electrolytes such as tetrabutylammonium perchlorate and the like.

Fatty acids having 11 to 30 carbon atoms, and salts and derivatives thereof are preferred as a crystalline compound, of which ester compounds of fatty acids and polyhydric alcohols are more preferred, and esters of glycerine and fatty acids represented by (a) to (c) indicated hereinbefore and serving as a hair growth effective ingredient are also more preferred.

The crystalline compounds may be used singly or in combination of two or more, and the amount is preferably at 0.001 to 40% by weight, more preferably 0.1 to 20% by weight, of the cosmetic hair preparation.

The cosmetic hair preparation of the invention is one obtained by blending at least one ester compounds selected from ester compounds of fatty acids having 2 to 10 carbon atoms and polyhydric alcohols, ester compounds of monobasic oxyacids and monohydric or polyhydric alcohols, and ester compounds of polybasic acids and monohydric or polyhydric alcohols with a cosmetic hair preparation containing a crystalline compound and a solvent capable of dissolving the crystalline compound. By blending the ester compound, it is possible to inhibit from crystallization and precipitation of the crystalline compounds at low temperatures.

Examples of the fatty acids having 2 to 10 carbon atoms include butyric acid, valeric acid, hexanoic acid, octanoic acid, decanoic acid and the like.

Examples of the monohydric alcohols include methyl alcohol, ethyl alcohol, propyl alcohol, isopropyl alcohol, butyl alcohol, isobutyl alcohol, amyl alcohol, caproyl alcohol, caprylyl alcohol, capryl alcohol, lauryl alcohol, myristyl alcohol, cetyl alcohol, stearyl alcohol, isostearyl alcohol, arachyl alcohol, behenyl alcohol, carnaubyl alcohol, ceryl alcohol, cholianyl alcohol, myricyl alcohol, melissyl alcohol, lacseryl alcohol, allyl alcohol, crotyl alcohol, 1-butenyl alcohol, 2-pentanol, 3-hexanol, 2-heptenol, 10-undecenol, 11-dodecenol, 12-tridecenol, oleyl alcohol, elaidyl alcohol, linoleyl alcohol, linolenyl alcohol, octyldodecanol, hexyldecanol, decyltetradecanol, ethylhexyl alcohol, triethylhexyl alcohol, cholesterol, phytosterol, benzyl alcohol, jojoba alcohol, and derivatives thereof. Of these, methyl alcohol, ethyl alcohol, propyl alcohol, isopropyl alcohol, butyl alcohol, isobutyl alcohol, amyl alcohol, caproyl alcohol, caprylyl alcohol, capryl alcohol, lauryl alcohol, myristyl alcohol, cetyl alcohol, stearyl alcohol, isostearyl alcohol, arachyl alcohol, behenyl alcohol, octyldodecanol, hexyldecanol, decyltetradecanol, ethylhexyl alcohol, triethylhexyl alcohol, cholesterol and phytosterol are preferred.

Examples of polyhydric alcohols include fructose, glucose, maltose, multitol, sucrose, xylitol, mannitol, sorbitol, erythritol, pentaerythritol, trehalose, mannotriose, threytol, amylolysis sugar reduced alcohol, glysolid, sorbitan, polyoxyethylene methyl glycoside, polyoxypropylene, methyl glycoside, trimethylpropanol, ethylene glycol, diethylene glycol, triethylene glycol, tetraethylene glycol, polyethylene glycol, propylene glycol, dipropylene glycol, polypropylene glycol, neopentyl glycol, glycerine, diglycerine, triglycerine, tetraglycerine, pentaglycerine, decaglycerine, polyglycerine, 3-methyl-1,3-butandiol, 1,3-butylene glycol, butylethylpropandiol, trimethylolpropane, and derivatives thereof. Of these, erythritol, pentaerythritol, trehalose, polyoxypropylene, polyethylene glycol, propylene glycol, dipropylene glycol, polypropylene glycol, neopentyl glycol, glycerine, diglycerine, triglycerine, tetraglycerine, pentaglycerine, decaglycerine, polyglycerine, 3-methyl-1,3-butandiol, 1,3-butylene glycol, butylethylpropandiol and trimethylolpropane are preferred.

The monobasic oxy acid is a compound having a hydroxyl group and one carboxyl group in the molecular structure. For the monobasic oxy acid, there are preferably used hydroxystearic acid, glycolic acid, lactic acid, glyceric acid and derivatives thereof.

The polybasic acid is a compound having two or more carboxyl groups in the molecular structure. Examples of the polybasic acids include citric acid, malic acid, tartaric acid, oxalic acid, malonic acid, succinic acid, glutaric acid, adipic acid, pimelic acid, suberic acid, azelaic acid, sebacic acid, 1,9-nanomethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicaroxylic acid, 1,14-tetradecamethylenedicarboxylic acid, 1,15-pentadecamethylenedicarboxylic acid, 1,16-hexadecamethylenedicarboxylic acid, 1,17-heptadecamethylenedicarboxylic acid, 1,18-octadecamethylenedicarboxylic acid, 1,19-nanodecamethylenedicarboxylic acid, 1,20-icosadecamethylenedicarboxylic acid, 1,21-henicosamethylenedicarboxylic acid, 1,22-docosamethylenedicarboxylic acid, 1,24-tetracosamethylenedicarboxylic acid, 1,28-octacosamethylenedicarboxylic acid, 1,32-dotriacontamethylenedicarboxylic acid, and derivatives thereof. Of these, citric acid, tartaric acid, oxalic acid, malonic acid, succinic acid, glutaric acid, adipic acid, and sebacic acid are preferred.

For the ester compounds of fatty acids having 2 to 10 carbon atoms and polyhydric alcohols include propylene glycol decanoate, neopentyl glycol dicanoate, pentaerythritol tetra-2-ethylhexanoate, propylene glycol monooctanoate, propylene glycol dioctanoate, trimethylolpropane tri-2-ethylhexanoate, glyceryl tri-2-ethylhexanoate, and 2-butyl-2-ethyl-1,3-propanediol 2-ethylhexanoate.

Examples of the ester compounds of monobasic oxy acids and mono- or polyhydric alcohols include 2-ethylhexyl hydroxystearate, choresteryl hydroxystearate, isostearyl lactate, and octyldodecyl lactate.

Examples of the ester compounds of polybasic acids and mono- or polyhydric alcohols include ethyl citrate, diethyl citrate, triethyl citrate, acetyltriethyl citrate, acetyltributyl citrate, dimethyl citrate, propyl citrate, bipropyl citrate, isopropyl citrate, diisopropyl citrate, 2-ethylhexyl citrate, isobutyl citrate, diisobutyl citrate, isostearyl citrate, diisostearyl citrate, octyl citrate, dioctyl citrate, 2-ethylhexyl citrate, tri-2-ethylhexyl citrate, ethyl malate, diethyl malate, methyl malate, dimethyl malate, propyl malate, dipropyl malate, isopropyl malate, diisopropyl malate, 2-ethylhexyl malate, isobutyl malate, diisobutyl malate, isostearyl malate, diisostearyl malate, octyl malate, dioctyl malate, 2-ethylhexyl malate, tri-2-ethylhexyl malate, ethyl oxalate, diethyl oxalate, methyl oxalate, dimethyl oxalate, propyl oxalate, dipropyl oxalate, isopropyl oxalate, diisopropyl oxalate, 2-ethylhexyl oxalate, isobutyl oxalate, diisobutyl oxalate, isostearyl oxalate, diisostearyl oxalate, octyl oxalate, dioctyl oxalate, 2-ethylhexyl oxalate, tri-2-ethylhexyl oxalate, ethyl tartarate, diethyl tartarate, methyl tartarate, dimethyl tartarate, propyl tartarate, dipropyl tartarate, isopropyl tartarate, diisopropyl tartarate, 2-ethylhexyl tartarate, isobutyl tartarate, diisobutyl tartarate, isostearyl tartarate, diisostearyl tartarate, octyl tartarate, dioctyl tartarate, 2-ethylhexyl tartarate, tri-2-ethylhexyl tartarate, ethyl maloate, diethyl maloate, methyl maloate, dimethyl maloate, propyl maloate, dipropyl maloate, isopropyl maloate, diisopropyl maloate, 2-ethylhexyl maloate, isobutyl maloate, diisobutyl maloate, isostearyl maloate, diisostearyl maloate, octyl maloate, dioctyl maloate, 2-ethylhexyl maloate, tri-2-ethylhexyl maloate, ethyl succinate, diethyl succinate, methyl succinate, dimethyl succinate, propyl succinate, dipropyl succinate, isopropyl succinate, diisopropyl succinate, 2-ethylhexyl succinate, diethyl hexyl succinate, isobutyl succinate, diisobutyl succinate, isostearyl succinate, diisostearyl succinate, octyl succinate, dioctyl succinate, 2-ethylhexyl succinate, tri-2-ethylhexyl succinate, ethyl glutarate, diethyl glutarate, methyl glutarate, dimethyl glutarate, propyl glutarate, dipropyl glutarate, isopropyl glutarate, diisopropyl glutarate, 2-ethylhexyl glutarate, isobutyl glutarate, diisobutyl glutarate, isostearyl glutarate, diisostearyl glutarate, octyl glutarate, dioctyl glutarate, 2-ethylhexyl glutarate, tri-2-ethylhexyl glutarate, ethyl adipate, diethyl adipate, methyl adipate, dimethyl adipate, propyl adipate, dipropyl adipate, isopropyl adipate, diisopropyl adipate, 2-ethylhexyl adipate, isobutyl adipate, diisobutyl adipate, isostearyl adipate, diisostearyl adipate, octyl adipate, dioctyl adipate, 2-ethylhexyl adipate, tri-2-ethylhexyl adipate, ethyl sebacate, diethyl sebacate, methyl sebacate, dimethyl sebacate, propyl sebacate, dipropyl sebacate, isopropyl sebacate, diisopropyl sebacate, 2-ethylhexyl sebacate, isobutyl sebacate, diisobutyl sebacate, isostearyl sebacate, diisostearyl sebacate, octyl sebacate, dioctyl sebacate, 2-ethylhexyl sebacate, and tri 2-ethylhexyl sebacate. Of these, triethyl citrate, acetyltriethyl citrate, acetyltributyl citrate, diisostearyl malate, diethylhexyl succinate, diisopropyl adipate, diisobutyl adipate, dioctyl adipate, tri-2-ethylhexyl adipate, diethyl sebacate, and diisopropyl sebacate are preferred.

In the present invention, it is preferred to use, in combination, two or more of members selected from ester compounds of fatty acids having 2 to 10 carbon atoms and polyhydric alcohols, ester compounds of monobasic oxy acids and mono- or polyhydric alcohols, and ester compounds of polybasic acids and mono- or polyhydric alcohols. Preferred combinations include combinations of two or more selected from dibasic acid esters such as an octanoic acid ester, a decanoic acid ester, a malic acid ester, a citric acid ester, a lactic acid ester, a hydroxystearic acid ester, an adipic acid ester and the like.

The amount of one or more of the esters compounds selected from ester compounds of fatty acids having 2 to 10 carbon atoms and polyhydric alcohols, ester compounds of monobasic oxy acid and mono- or polyhydric alcohols, and polybasic acids and mono- or polyhydric alcohols is generally 0.01 to 60% by weight, preferably 0.1 to 40% by weight and more preferably 0.5 to 30% by weight of the cosmetic hair preparation. If the amount is too small, a satisfactory effect may not be obtained. Larger amounts may be poor in economy, in some cases, inconvenient to feeling to use such as hair stickiness and the like.

The solvent used in the cosmetic hair preparation of the present invention is not critical in type, and water and organic solvents are all usable singly or in combination of two or more so far as they are capable of dissolving the crystalline compound and such an ester compound as set out above. An organic solvent may be either hydrophilic or lipophilic, and a non-aqueous solvent whose water content is at 5% by weight or below is favorably usable. For such a non-aqueous solvent includes methyl alcohol, ethyl alcohol, propyl alcohol, isopropyl alcohol, n-paraffin, benzene, hexane, chloroform and the like. Of these, the use of ethyl alcohol is preferred from the standpoint of safety.

In the cosmetic hair preparation of the present invention, there may be further added, aside from those ingredients set forth hereinabove, arbitrary ingredients depending on the purposes in use. For instance, there may be blended medicinal ingredients such as polyhydric alcohols, surface active agents, oils and fats, vitamins, hormones, vasolidators, amino acids, antiinflammatory medications, skin function enhancers, keratin improvers and the like, and fragrances. For the cosmetic hair preparation, hair tonic, hair liquid, hair lotion, hair spray, hair growth agent, sculp care and the like are mentioned, of which the hair growth agent is preferred.

If a fragrance is added in the cosmetic hair preparation of the present invention, the fragrances used are accorded to the fragrances and fragrance compositions set out in Japanese Patent Laid-Open No. 2003-95895. If a fragrance composition is formulated, the fragrance composition is preferably formulated in an amount of 0.00001 to 50% by weight, more preferably 0.0001 to 30% by weight, relative to the total of the cosmetic hair preparation.

The cosmetic hair preparation of the invention can be prepared based on a usual manner and can be used based on ordinary procedures of individual preparations.

The cosmetic hair preparation of the invention can inhibit a crystalline compound from being crystallized and precipitated at low temperatures and shows excellent effects of improving solubility and low temperature stability. In this way, when stored at a low temperature of about −5° C. over a long time, neither turbidity of the solution nor precipitation of the crystalline compound occurs, thus being excellent in low temperature stability.

### EXAMPLES

Examples and Comparative Examples are shown below to illustratively explain the invention, although the invention should not be construed as limiting to the following examples. It will be noted that unless otherwise indicated, “%” in composition is by weight.

Cosmetic hair preparations having formulations indicated in Tables 1, 2 were prepared and the days of stabilization and low temperature stability were assessed according to the following methods to check an inhibition effect of crystallization and also evaluate stickiness to check a feeling to use at the point of use of preparations. The results are both shown in Tables 1 and 2.

<Evaluation Methods of Days of Stabilization and Low Temperature Stability>

About 20 ml of a sample to be tested was placed in a 50 ml transparent glass bottle and stored at a temperature-controlled room at −5° C. to determine days for stabilization by subtracting one day from days at which crystals precipitated. It will be noted that the results are indicated in terms of days obtained by half adjusting an average value of the results of three measurements. The low temperature stability was evaluated as ⊚ when the days for stabilization were 40 days or over, as ◯ in case of 20 to 39 days, and as Δ in case of 10 to 19 days, and as X in case of 9 days or below.

<Evaluation of Stickiness>

About 2 g of a sample to be tested was applied uniformly to an entire hairpiece (Beaulax Co., Ltd.) for evaluation into which human hair was implanted, and stickiness was subjected to sensory evaluation made by five professional panelists based on the following evaluation standards to obtain an average value of the five panelists thereby providing ratings on the basis of the following comprehensive valuation criterion.

Evaluation Standards

5: good (little stickiness)
4: slightly good (reduced degree of stickiness)
3: moderate (no problem from the standpoint of a commercial value)
2: slightly bad (some degree of stickiness with a problem involved in commercial value)
1: bad (appreciable degree of stickiness)
   Comprehensive Valuation Criterion (the Average Value of the Five Panelists)
4 or over: ⊚
from 3 to smaller than 4: ◯
from 2 to smaller than 3: Δ

from 1 to smaller than 2: X

TABLE 1

| | Example | | | | | | | |
|---|---|---|---|---|---|---|---|---|
| Composition (%) | 1 | 2 | 3 | 4 | 5 | 6 | 7 | 8 |
| Glyceride pentadecanoate | 2.0 | — | — | 2.0 | 2.0 | 2.0 | 2.0 | 2.0 |
| Glyceride tridecanoate | — | 2.0 | — | — | — | — | — | — |
| Ethyl pentadecanoate | — | — | 2.5 | — | — | — | — | — |
| Propylene glycol monooctanoate | 20.0 | — | — | — | — | 10.0 | 5.0 | 2.5 |
| Isostearyl malate | — | 15.0 | — | — | 10.0 | — | — | — |
| 2-ethylhexyl hydroxy-stearate | — | — | 20.0 | — | — | — | — | — |
| Triethylhexyl citrate | — | — | — | 15.0 | 10.0 | 10.0 | — | 2.5 |
| Butyl myristate | — | — | — | — | — | — | — | — |
| Isopropyl myristate | — | — | — | — | — | — | — | — |
| Isopropyl palmitate | — | — | — | — | — | — | — | — |
| Ethyl alcohol | balance | balance | balance | balance | balance | balance | balance | balance |
| Total | 100.0 | | | | | | | |
| Days for stabilization (days) | 29 | 33 | 31 | 30 | 43 | 45 | 20 | 25 |
| Low temperature stability | ◯ | ◯ | ◯ | ◯ | ⊚ | ⊚ | ◯ | ◯ |
| Feeling in use | ⊚ | ◯ | ◯ | ⊚ | ⊚ | ⊚ | ⊚ | ⊚ |
| (no degree of stickiness) | | | | | | | | |

TABLE 2

| | Comparative Example | | | |
|---|---|---|---|---|
| Composition (%) | 1 | 2 | 3 | 4 |
| Glyceride pentadecanoate | 2.0 | — | — | 2.0 |
| Glyceride tridecanoate | — | 2.0 | — | — |
| Ethyl pentadecanoate | — | — | 2.0 | — |
| Propylene glycol monooctanoate | — | — | — | — |
| Isostearyl malate | — | — | — | — |
| 2-ethylhexyl hydroxystearate | — | — | — | — |
| Triethylhexyl citrate | — | — | — | — |
| Butyl myristate | 20.0 | — | — | 10.0 |
| Isopropyl myristate | — | 20.0 | — | 10.0 |
| Isopropyl palmitate | — | — | 15.0 | 10.0 |
| Ethyl alcohol | balance | balance | balance | balance |
| Total | 100.0 | | | |
| Days for stabilization (days) | 3 | 5 | 9 | 18 |
| Low temperature stability | X | X | X | Δ |
| Feeling in use | ◯ | ⊚ | ◯ | ⊚ |
| (no degree of stickiness) | | | | |

The following cosmetic hair preparations were prepared. The respective preparations were prepared based on ordinary procedures unless otherwise described.

### Example 9

TABLE 3
Pilatory

| | wt % |
|---|---|
| Glyceride monopentadecanoate | 3.0 |
| DL-α-tocopherol acetate | 0.1 |
| β-Glycyrrhetic acid | 0.05 |
| Propylene glycol monooctanoate | 20.0 |
| 2-Ethylhexyl hydroxystearate | 10.0 |
| Isopropylmethylphenol | 0.1 |
| Ethyl oleate | 2.0 |
| Coconut oil fatty acid sorbitan ester | 1.0 |
| Sucrose myristic acid ester | 0.5 |
| Lauryl dimethylaminoacetic betaine | 0.5 |
| Ampholytic polymer *1 | 0.2 |
| Succinic acid | 0.3 |
| Coleus extract | 1.0 |
| L-Menthol | 0.3 |
| Fragrance *2 | 0.1 |
| 99.5% ethyl alcohol | balance |
| Total | 100.0 |

*1 N-methacryloyloxyethyl-N,N-dimethylammonium α-N-methylcarboxybetaine/alkyl methacrylate copolymer
*2 Composition B shown in Table 2 of Japanese Patent Laid-Open No. 2003-113019

### Example 10

TABLE 4
Hair growth spray

| | wt % |
|---|---|
| Glyceride monopentadecanoate | 1.0 |
| Propylene glycol monooctanoate | 15.0 |
| Octyldodecyl lactate | 10.0 |
| Sorbitan laurate | 0.5 |
| Ampholytic polymer *1 | 0.2 |
| Ethyl oleate | 0.5 |
| Citric acid | 0.3 |
| Sucrose lauric acid ester | 0.5 |
| Fragrance *2 | 0.1 |
| L-Menthol | 0.1 |
| 99.5% ethyl alcohol | balance |
| Total | 100.0 |
| Above stock solution | 80.0 |
| LPG | 20.0 |
| Total | 100.0 |

*1 N-methacryloyloxyethyl-N,N-dimethylammonium α-N-methylcarboxybetaine/alkyl methacrylate copolymer
*2 Composition C shown in Table 2 of in Japanese Patent Laid-Open No. 2003-113019

### Example 11

TABLE 5
Hair growth tonic

| | wt % |
|---|---|
| Glyceride monopentadecanoate | 2.0 |
| Propylene glycol monooctanoate | 10.0 |
| Diisostearyl malate | 10.0 |
| Propylene glycol decanoate | 5.0 |
| POE (8 moles) oleyl alcohol ether | 1.5 |
| Glycerine | 3.0 |
| L-Menthol | 0.1 |
| Methylparaben | 0.1 |
| Fragrance* | 0.3 |
| 99.5% ethylene alcohol | 60.0 |
| Purified water | balance |
| Total | 100.0 |

*Composition D shown in Table 2 of Japanese Patent Laid-Open No. 2003-113019

### Example 12

TABLE 6
Hair growth lotion

| | wt % |
|---|---|
| Glyceride monopentadecanoate | 1.0 |
| DL-α-tocopherol acetate | 0.5 |
| Propylene glycol monooctanoate | 15.0 |
| Isostearyl lactate | 10.0 |
| Sucrose myristic acid ester | 0.5 |
| POE (40) hardened castor oil | 0.5 |
| Citric acid | 0.1 |
| L-Menthol | 0.1 |
| Fragrance* | 0.1 |
| 99.5% ethyl alcohol | balance |
| Total | 100.0 |

*Composition D shown in Table 2 of Japanese Patent Laid-Open No. 2003-113019

### Example 13

TABLE 7
Hair growth lotion

| | wt % |
|---|---|
| Glyceride monopentadecanoate | 2.0 |
| Diethylhexyl succinate | 10.0 |
| Octyldodecyl lactate | 10.0 |
| Diethyl sebacate | 5.0 |
| Sorbitan laurate | 0.5 |
| DL-α-tocopherol acetate | 0.5 |
| Olive oil | 1.0 |
| Succinic acid | 0.2 |
| Fragrance* | 0.1 |
| 80% ethyl alcohol | balance |
| Total | 100.0 |

*Composition C shown in Table 2 of Japanese Patent Laid-Open No. 2003-113019

### Example 14

TABLE 8
Hair growth shampoo

| | wt % |
|---|---|
| Glyceride monopentadecanoate | 4.0 |
| Triethylhexyl citrate | 10.0 |
| Polypropylene glycol succinate | 10.0 |
| Diisobutyl adipate | 5.0 |
| Sodium lauryl ether sulfate | 10.0 |
| Sodium α-olefinsulfonate | 5.0 |
| Fatty acid diethanolamide | 5.0 |
| Alklyamidopropyl betaine | 5.0 |
| Dimethylsiloxane | 3.0 |
| Cationized cellulose | 0.5 |
| Ethylene glycol distearate | 2.0 |
| Sodium benzoate | 0.8 |
| Fragrance* | 0.1 |
| Purified water | balance |
| Total | 100.0 |

*Composition E shown in Table 2 of Japanese Patent Laid-Open No. 2003-113019

### Example 15

TABLE 9
Hair growth rinse

| | wt % |
|---|---|
| Glyceride monopentadecanoate | 0.5 |
| Octyldecyl lactate | 10.0 |
| Triethylhexyl citrate | 10.0 |
| Diisopropyl dilinolate | 5.0 |
| Stearyltrimethylammonium chloride | 1.0 |
| Distearyldimethylammonium chloride | 0.5 |
| Setostearyl alcohol | 3.0 |
| POE(5) stearyl ether | 1.5 |
| Liquid paraffin | 1.0 |
| Cyclic silicone (decamethylpentasiloxane) | 0.5 |
| Paraben | 0.1 |
| Fragrance* | 0.1 |
| Purified water | balance |
| Total | 100.0 |

*Composition E shown in Table 2 of Japanese Patent Laid-Open No. 2003-113019

### Example 16

Pilatory Cream

A and B indicated below were, respectively, dissolved at 70° C., and A was added to B and uniformly emulsified. While cooling, C was added to prepare a pilatory cream.

TABLE 10

| | | wt % |
|---|---|---|
| A | Oil phase portion | |
| | Glyceride monopentadecanoate | 4.0 |
| | Ethyl oleate | 2.0 |
| | 2-Ethylhexyl hydroxystearate | 10.0 |
| | Triethylhexyl citrate | 15.0 |
| | Diisobutyl adipate | 5.0 |
| | Octyldodecyl myristate | 1.5 |
| | Liquid paraffin | 1.0 |
| | Polyethylene glycol (40 EO) monostearate | 3.0 |
| | Coconut oil fatty acid sorbitan | 2.0 |
| | Glycerine monostearate | 1.0 |
| | Setostearyl alcohol | 1.0 |
| | Pantotenyl ethyl ether | 0.5 |
| | Propylparaben | 0.1 |
| B | Aqueous phase portion | |
| | 1,3-Butylene glycol | 2.5 |
| | Dipropylene glycol | 2.5 |
| | Methylparaben | 0.25 |
| | Dipotassium glycyrrhizinate | 0.2 |
| | Placenta liquid | 0.2 |
| C | Fragrance* | 0.1 |
| | Purified water | balance |
| | Total | 100.0 |

*Composition B shown in Table 2 of Japanese Patent Laid-Open No. 2003-113019

### Example 17

TABLE 11
Hair growth drug

| | wt % |
|---|---|
| Glyceride monopentadecanoate | 2.5 |
| DL-α-tocopherol acetate | 0.2 |
| Diisostearyl malate | 15.0 |
| Polypropylene glycol succinate | 5.0 |
| Diisobutyl adipate | 10.0 |
| Pentaglycerine monomyristate | 1.0 |
| Sorbitan laurate | 0.5 |
| L-Menthol | 0.05 |
| Benzyl nicotinate | 0.001 |
| Licorice extract | 0.2 |
| Methylparaben | 0.05 |
| Fragrance* | 0.1 |
| 95% ethyl alcohol | balance |
| Total | 100.0 |

*Composition C shown in Table 2 of Japanese Patent Laid-Open No. 2003-113019

### Example 18

TABLE 12
Hair growth liquid

| | wt % |
|---|---|
| Glyceride monopentadecanoate | 1.0 |
| Stearyl glycyrrhizinate | 0.2 |
| Propylene glycol monooctanoate | 5.0 |
| Diisostearyl malate | 5.0 |
| Triethylhexyl citrate | 5.0 |
| Diisobutyl adipate | 2.0 |
| Sucrose lauric acid ester | 0.5 |
| Hexaglycerine dilaurate | 0.5 |
| POP (5) butyl ether | 20.0 |
| Lanoline | 1.0 |
| Carboxy vinyl polymer | 0.8 |
| Oxybenzone | 0.1 |
| Methylparaben | 0.1 |
| Triisopropanolamine | 0.1 |
| Fragrance* | 0.1 |
| 95% ethyl alcohol | balance |
| Total | 100.0 |

*Composition B shown in Table 2 of Japanese Patent Laid-Open No. 2003-113019

### Example 19

TABLE 13
Hair growth agent

| | wt % |
|---|---|
| Glyceride pentadecanoate | 2.0 |
| Tocopherol acetate | 0.1 |
| 6-Benzylaminopurine | 0.5 |
| Ethyl oleate | 3.0 |
| Octyldodecyl lactate | 3.0 |
| Propylene glycol monooctate | 0.5 |
| Sucrose fatty acid ester | 0.8 |
| L-Menthol | 0.2 |
| Concentrated glycerine | 0.5 |
| Succinic acid | 0.1 |
| 99.5% ethyl alcohol | balance |
| Total | 100.0 |
| Above stock solution | 60.0 |
| DME | 40.0 |
| Total | 100.0 |

### Example 20

TABLE 14
Hair growth agent

| | wt % |
|---|---|
| Glyceride pentadecanoate | 2.0 |
| 6-Benzylaminopurine | 0.3 |
| Ethyl oleate | 3.0 |
| Octyldodecyl lactate | 1.0 |
| Propylene glycol monooctanoate | 1.0 |
| Sorbitan laurate | 2.5 |
| Lauryldimethylaminoacetic acid betaine | 0.3 |
| L-Menthol | 0.2 |
| Citric acid | 0.2 |
| Purified water | 10.0 |
| 99.5% ethyl alcohol | balance |
| Total | 100.0 |

The hair growth agents of Examples 9 to 20 were all excellent in low temperature stability and exhibited good feeling upon using the hair growth agents.

The starting materials used in the Examples and Comparative Examples are indicated below.

TABLE 15

| | | Manufacturer |
|---|---|---|
| Name of starting material | Commercial name | for starting material |
| Glyceride monopentadecanoate | Glyceride pentadenoate | Lion Chemicals Corporation |
| Ethyl alcohol | 99-proof, first grade, | Shinwa Alcohol Industry Co., |
| | specified alcohol | Ltd. |
| DL-α-tocopherol acetate | DL-α-tocopherol acetate | DSM Nutrition Japan K.K. |
| β-Glycyrrhetic acid | Glycyrrhetic acid | Maruzen Pharmaceuticals Co., |
| | | Ltd. |
| Isopropylmethylphenol | Isopropylmethylphenol | Tokyo Kasei Kogyo Co., Ltd. |
| Ethyl oleate | Ethyl oleate | NOF Corporation |
| Sucrose myristic acid ester | M-1695 | Mitsubishi Kagaku Foods |
| | | Corporation |
| Luarydimethylaminoacetic acid | AM-301 | Nikko Chemicals Co., Ltd. |
| betaine | | |
| Ampholytic polymer | Yukaformer 201 | Mitsubishi Chemical Corporation |
| Succinic acid | Succinic acid | Kawasaki Kasei Chemicals Ltd. |
| Coleus extract | Coleus extract | Toyotama International Inc. |
| L-Menthol | L-Menthol | Takasago International |
| | | Corporation |
| Sorbitan laurate | Nonion LP-20R | NOF Corporation |
| Citric acid | Citric acid | Fuso Chemical Co., Ltd. |
| Sucrose lauric acid ester | L-595 | Mitsubishi Kagaku Foods |
| | | Corporation |
| LPG | LPG | Daizo Corporation |
| POE (8 mols) oleyl alcohol | Nonion E-202S | NOF Corporation |
| ether | | |
| Glycerine | Glycerine | New Japan Chemical Co., Ltd. |
| POE (40) hardened castor oil | HCO-40 | Nikko Chemicals Co., Ltd. |
| Olive oil | Olive oil | Kenei Pharmaceutical Co., Ltd. |
| Sodium lauryl ether sulfate | Sodium POE (2) lauryl ether | Nikko Chemicals Co., Ltd. |
| | sulfate aqueous solution | |
| Sodium α-olefinsulfonate | K Liporan | Lion Chemicals Corporation |
| Fatty acid diethanolamide | Staform DFC | NOF Corporation |
| Alkylamidopropyl betaine | Nissan Anon BDF-S | NOF Corporation |
| Dimethylsiloxane | SH200C-1cs | Dow Corning Toray Co., Ltd. |
| Cationized cellulose | LeoGuard GP | Lion Chemicals Corporation |
| Sodium benzoate | Amisoft LK-22 | Ajinomoto Co., Inc. |
| Stearyltrimethylammonium | Arquad T-800 | Lion Chemicals Corporation |
| chloride | | |
| Distearyldimethylammonium | CA-3475V | Nikko Chemicals Co., Ltd. |
| chloride | | |
| Setostearyl alcohol | NAA-48 | NFO Corporation |
| POE (5) stearyl ether | 605 | Nihon-Emulsion Co., Ltd. |
| Cyclic silicone | SH245 | Dow Corning Toray Co., Ltd. |
| (decamethylpentasiloxane) | | |
| Paraben | Methyl paraoxybenzoate | Yoshitomiseiyaku Corporation |
| Liquid paraffin | Daphne Oil CP | Idemitsu Kosan Co., Ltd. |
| Polyethylene monostearate | MYS-40 | Nikko Chemicals Co., Ltd. |
| Coconut oil fatty acid | SC-10 | Nikko Chemicals Co., Ltd. |
| sorbitan | | |
| Glycerine monostearate | MGS-F20 | Nikko Chemicals Co., Ltd. |
| Setostearyl alcohol | Lannette O | Cognis Japan Ltd. |
| Pantotenyl ethyl ether | Pantotenyl ethyl ether | Daiichi Fine Chemical Co., Ltd. |
| 6-Benylaminopurine | CTP | Sansho Seiyaku Co., Ltd. |

TABLE 16

| | | |
|---|---|---|
| Propylparaben | Propyl paraoxybenzoate | Midori Kagaku Co., Ltd. |
| 1,3-butylene glycol | 1,3-butylene glycol (85%) | Daicel Chemical Industries |
| | | Ltd. |
| Dipropylene glycol | Dipropylene glycol | Seiko Bussan K.K. |
| Methylparaben | Methyl paraoxybenzoate | Yoshitomiseiyaku Corporation |
| Dipotassium glycyrrhizinate | Glycyrrhitin K2 | Maruzen Pharmaceuticals Co., |
| | | Ltd. |
| Placenta liquid | Pharconix BPS | Ichimaru Pharcos Co., Ltd. |
| Pentaglycerine monomyristate | Sunsoft A141E | Taiyo Kagaku Co., Ltd. |
| Sorbitan laurate | SL-10 | Nikko Chemicals Co., Ltd. |
| Benzyl nicotinate | Benzyl nicotinate | Kotobuki Chemical Co., Ltd. |
| Licorice extract | Licorice extract | Maruzen Pharmaceuticals Co., |
| | | Ltd. |
| Stearyl glycyrrhizinate | CO-gretinol | Maruzen Pharmaceuticals Co., |
| | | Ltd. |
| Lanolin | LANOLIN | Nippon Fine Chemical Co., Ltd. |
| Carboxy vinyl polymer | Carbopole 981 | Nikko Chemicals Co., Ltd. |
| Oxybenzone | Uvinul M-40 | BASF Japan Ltd. |
| Diethylhexyl sebacate | KAK DIOS | Kokyu Alcohol Kogyo Co., Ltd. |
| Isostearyl lactate | CETIOL ISL | Cognis Japan Ltd. |
| Diethyl sebacate | DES-SP | Nikko Chemicals Co., Ld. |
| Octyldodecyl lactate | Cosmole 13 | Nisshin OilliO Group, Ltd. |
| 2-Ethylhexyl hydroxystearate | Lisocastor IOHS | Kokyu Alcohol Kogyo Co., Ltd. |
| Triethylhexyl citrate | TOC | Nikko Chemicals Co., Ltd. |
| Octyldodecyl myristate | ODM-100 | Nikko Chemicals Co., Ltd. |
| Polypropylene glycol | Cosmole 102 | Nisshin OilliO Group, Ltd. |
| succinate | | |
| Diisobutyl adipate | KAK DIBA | Kokyu Alcohol Kogyo Co., Ltd. |
| Propylene glycol | Sefsol 218 | Nikko Chemicals Co., Ltd. |
| monooctanoate | | |
| Diisostearyl malate | DISM | Nikko Chemicals Co., Ltd. |
| Butyl myristate | BM | Nikko Chemicals Co., Ltd. |
| Isopropyl myristate | IPM-EX | Nikko Chemicals Co., Ltd. |
| Isopropyl palmitate | IPP-EX | Nikko Chemicals Co., Ltd. |

1. A cosmetic hair preparation comprising a crystalline compound, a solvent capable of dissolving the crystalline compound, and at least one ester compound selected from the group consisting of ester compounds of fatty acids having 2 to 10 carbon atoms and polyhydric alcohols, ester compounds of monobasic oxyacids and monohydric or polyhydric alcohols, and ester compounds of polybasic acids and monohydric or polyhydric alcohols.

2. The cosmetic hair preparation according to claim 1, wherein the crystalline compound is selected from (i) fatty acids having 11 to 30 carbon atoms, and salts and derivatives thereof, (ii) fatty alcohols, and (iii) ether compounds of fatty alcohols and compounds having hydroxyl group.
